(19) United States
(12) Patent Application Publication
(10) Pub. No.: US 2018/0272071 A1
(43) Pub. Date: Sep. 27, 2018

(54) System and Method for Associating a Syringe with a Corresponding Vial and Medicinal Composition

(71) Applicant: Yong Shin Chen, Glendora, CA (US)

(72) Inventor: Yong Shin Chen, Glendora, CA (US)

(21) Appl. No.: 15/467,181

(22) Filed: Mar. 23, 2017

(51) Int. Cl. A61M 5/32; A61J 1/20; A61M 5/34; G09F 3/10; G09F 3/02

(52) U.S. Cl. CPC A61M 5/3202; A61M 5/32; A61J 1/2096; A61M 2005/3125; G09F 3/10; G09F 3/02; A61M 5/347

(57) ABSTRACT

A system and method associates a syringe and an attached spike with a corresponding vial and the medicinal composition contained in the vial. The system includes a labeled seal that detachably attaches to a spike and is removed for attachment to syringe. The labeled seal has an identifier, such as a color-coding, a tactile, or textual indicia. These identifiers on the labeled seal matches identifiers on a vial label that attaches to a vial containing the medicinal composition. The matching identifiers on the labeled seal and the vial label enable association between the syringe and the vial, so as to utilize the appropriate medicinal composition with the syringe. The identifier on the labeled seal is also indicative of the medicinal composition in the vial. A second label locks between the syringe and spike. The second label also serves to match the syringe with the corresponding vial and medicinal composition.

### FIELD OF THE INVENTION

The present invention relates generally to a system and method for associating a syringe with a corresponding vial and medicinal composition. More so, the present invention relates to a system and method that enables quick, efficient matching of medical syringes with corresponding drug vials and the fluid drug contained therein; whereby the system provides a vial containing a medicinal composition; a syringe having a male lock fitting; a spike having a cooperating female lock fitting and adapted to detachably attach to the syringe; a vial label; a labeled seal corresponding to the vial label and defined by at least one of the following: a colored panel, a tactile panel, and a textual indicia panel; and a second label corresponding to the vial label; whereby the vial label attaches to the vial; whereby the labeled seal detachably attaches to the spike and the syringe, so as to associate the syringe with a corresponding vial; whereby the second label attaches to the spike; whereby coupling the female lock fitting of the spike to the male lock fitting of the syringe fastens the second label to the syringe, so as to associate the syringe with the corresponding vial.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, medicinal compositions and drugs in liquid form, especially those used for subcutaneous intravenous injection, are distributed in pre-measured amounts and concentrations in glass or plastic vials having crimped metal seals that secure a rubber septum over the opening of the vial. The sterility or microbial integrity of the liquid contents of the vial is maintained as long as the seal remains in place and the septum is not penetrated by a non-sterile object.

In order to use the medication, a needle or plastic spike is affixed to a syringe, the plunger of the syringe is withdrawn to a volume that is roughly equivalent to the volume of the liquid that is desired to be transferred to the syringe, the needle or spike is used to pierce the septum, the vial is inverted to place the liquid contents in contact with the needle and the septum, the plunger of the syringe is depressed to inject air into the vial.

Often, this creates positive pressure in the vial, and then the plunger is again withdrawn while keeping the point of the needle submerged in the liquid, to draw the desired volume of liquid into the barrel of the syringe. Withdrawal of this volume of liquid equalizes pressure within the vial. The needle is then withdrawn from the septum, which re-seals, and the desired subcutaneous injection, or other disposition of the liquid, can take place.

Unfortunately, it requires multiple medicinal to treat a patient. Some medicinal fluids have similar remedial and/or chemical properties, whereas others can have quite different remedial effects. In some cases the use and/or mixing of medicinal fluids have divergent qualities that can be dangerous to the patient. During a particular medical procedure where multiple medicinal fluids, containers and medical syringes are utilized, a practitioner can become confused or unsure as to what medicinal fluid is contained in a particular medical syringe. Therefore, the syringes and vials containing medicinal compositions are labeled to maintain integrity and avoid cross-contamination of medicinal compositions between different syringes and needles and to avoid mistakes of giving the wrong drugs to patients.

This labeling procedure requires the medical practitioner to take many steps between the patient and the dispenser where the labels are located. Typically, a hospital may go through 500 of these syringe spikes per day. Thus, even if five seconds of time are saved walking, throughout the course of a day, it saves a much larger amount of time.

Other proposals have involved labeling syringes and medicinal compositions in vials. The problem with these syringe labeling methods is that they do not allow the practitioner to label without walking many steps, and they are not always secured to the syringe properly. Even though the above cited syringe labeling methods meet some of the needs of the market, a system and method that works to associate a syringe and an attached spike with a corresponding vial and the medicinal composition contained in the vial, though detachable attachment of a labeled seal and a second seal that matches identifiers on a vial label adhered to a vial containing the medicinal composition, is still desired.

### SUMMARY

Illustrative embodiments of the disclosure are generally directed to a system and method for associating a syringe with a corresponding vial and the medicinal composition contained therein. The system and method is effective for quickly associating a syringe and an attached spike with a corresponding vial and the medicinal composition contained therein, such as a drug vial containing a liquid drug. The syringe carries a labeled seal defined by at least one identifier that matches an identifier on a vial label that attaches to the vial. A matching identifier indicates that the syringe and attached spike are operational with the medicinal composition contained in the vial.

The system may include a syringe with an attached labeled seal. The labeled seal is defined by at least one identifier, such as a colored panel, a tactile panel, or a textual panel. The labeled seal matches the color, text, or tactile characteristics of a vial label that attaches to a vial containing a medicinal composition. The matching identifier on the labeled seal and the vial label allow a practitioner to quickly and effectively associate the syringe with a corresponding vial to access the appropriate medicinal composition for the syringe. In one embodiment, the identifier on the labeled seal is also indicative of the medicinal composition in the vial.

The system and method may be efficacious for enabling quick, efficient matching of medical syringes with corresponding drug vials containing a specific medicinal composition to be used with the syringe and attached spike. The present disclosure may be helpful in surgical and medical treatment environments where multiple syringes with attached spikes, and multiple vials containing different medicinal compositions are utilized during the course of a medical procedure. The present disclosure helps to minimize the potential for error associated with the delivery of medicinal compositions by providing an organizational system and method to quickly and effectively associate a syringe with the corresponding vial and medicinal composition contained therein.

The system may include: a vial containing a medicinal composition; a syringe having a male lock fitting; a spike having a cooperating female lock fitting and adapted to detachably attach to the syringe for administering the medicinal composition; a vial label defined by at least one identifier and attached to the vial; a labeled seal detachably attached to the syringe and corresponding to the vial label; and a second label attached to the syringe and corresponding to the vial label.

In one embodiment, the vial label attaches to the vial. The vial label comprises at least one identifier from at least one of the following: a colored panel, a tactile panel, and a textual indicia panel. In one embodiment, the identifier is a color that corresponds to a medicinal composition contained in the vial.

The labeled seal is configured to protectively seal the spike during storage, and also to detach from the spike container and attach to the syringe when the spike and syringe are coupled together for operation, i.e., administering the medicinal composition. Similar to the vial label, the labeled seal comprises the at least one identifier. The at least one identifier on the labeled seal enables the syringe to be associated with a matching identifier on the vial label.

Further, a second label attaches to both the spike and the syringe. Specifically, coupling the female lock fitting of the spike to the male lock fitting of the syringe works to fasten and display the second band/label on the syringe by pressing and rotation of the syringe into the spike. The second label is visible from the syringe when the spike and syringe are coupled together. Similar to the vial label and the labeled seal, the second label comprises the at least one identifier. In this manner, the syringe may be associated with a corresponding vial, and medicinal composition contained therein, based on the association between matching identifiers on the second label and the vial label. In emergency situation, a syringe can be identified by the label screwed onto the Lock tip and syringe and therefore forgoes the first label that is attached to the vial or spike container.

In one aspect, a system for associating a syringe with a corresponding vial and medicinal composition, comprises:
a vial containing a medicinal composition;
a syringe comprising a male lock fitting;
a spike or needle comprising a cooperating female lock fitting adapted to couple to the male lock fitting of the syringe, which is packaged sterilely in container;
whereby the coupled syringe and the spike work to administer the medicinal composition;
a vial label defined by at least one identifier, the vial label being attached to the vial;
a labeled seal defined by the at least one identifier, the labeled seal being detachably attached to the spike container or the syringe,
whereby the labeled seal associates the syringe to a corresponding vial based on the at least one identifier; and
a second label defined by the at least one identifier, the second label being attached to the syringe, or the spike, or both,
whereby the second label associates the syringe to the corresponding vial based on the at least one identifier.

In another aspect, the at least one identifier includes at least one of the following: a colored panel, a tactile panel, and a textual indicia panel.

In another aspect, the colored panel includes at least one of the following: a white color, a yellow color, an orange color, a green color, and a blue color.

In another aspect, the medicinal composition includes at least one of the following: Pitocin, Propofol, Versed, Atropine, and Sublimaze.

In another aspect, the tactile panel includes Braille.

In another aspect, the vial comprises a drug vial.

In another aspect, the male lock fitting and the female lock fitting comprise a Luer lock.

In another aspect, the male lock fitting and the female lock fitting are threaded and have a rotatable relationship.

In yet another aspect, syringe and the spike are in fluid communication.

In yet another aspect, labeled seal comprises a cover sized and dimensioned to substantially encapsulate the spike.

In yet another aspect, the cover of the labeled seal comprises an adhesive portion.

In yet another aspect, the labeled seal is adapted to form a rolled configuration, and to be perforated into multiple equally-sized segments.

In yet another aspect, the system comprises multiple spikes or its container having different identifiers and segregated based on the identifier in a container.

One objective of the present invention is to provide a system and method that enables quick, efficient matching of medical syringes and spikes with corresponding drug vials and the fluid drug contained therein.

Another objective is to allow medical practitioners, including doctors and nurses, to save the step of walking to a tape dispenser to obtain a label for the syringe, as they can just remove the labeled seal from the spike and adhere it to the syringe with the correct label color all at once.

Another objective is to save time, even a small amount of time labeling syringes and identifying the correct medicinal composition to use with the labeled syringe and spike, even as little as five seconds, throughout the course of a day.

Yet another objective is to provide the labeled seal on hand as the spike is attached to the syringe, so as to assist the medical practitioner in remembering to adhere the labeled seal to the syringe immediately.

Yet another objective is to minimize the chance that the medical practitioner forgets to walk to the label dispenser to access a label for the syringe while leaving the syringe unattended and unlabeled.

Yet another objective is to provide color coded identifiers for matching the syringe to the appropriate medicinal composition in the corresponding vial.

Yet another objective is to provide Braille identifiers for matching the syringe to the appropriate medicinal composition in the corresponding vial.

Yet another objective is to provide nomenclature terms as identifiers for matching the syringe to the appropriate medicinal composition in the corresponding vial.

Yet another objective is to add an additional layer of labeling security through use of a second label that is secured in a Luer lock between the male lock fitting of the syringe, and the female lock fitting of the spike.

Yet another objective is to provide an inexpensive to manufacture system and method for labeling syringes and identifying the corresponding vial containing the medicinal composition for the syringe.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a perspective view of an exemplary system for associating a syringe with a corresponding vial and medicinal composition, in accordance with an embodiment of the present invention;

FIG. 2 illustrates a perspective view of an exemplary syringe, in accordance with an embodiment of the present invention;

FIG. 3 illustrates a perspective view of an exemplary spike, in accordance with an embodiment of the present invention;

FIG. 4 illustrates a perspective view of a syringe coupled to a spike, in accordance with an embodiment of the present invention;

FIG. 5 illustrates a perspective view of an exemplary labeled seal covering a spike, in accordance with an embodiment of the present invention;

FIG. 6 illustrates a perspective view of a labeled seal adhering to a syringe, in accordance with an embodiment of the present invention;

FIG. 7 illustrates a perspective view of three types of labeled seals, in accordance with an embodiment of the present invention;

FIG. 8 illustrates a perspective view of an exemplary second label fastened between a syringe and a spike through a Luer lock, in accordance with an embodiment of the present invention;

FIG. 9 illustrates a flowchart of an exemplary method for associating a syringe with a corresponding vial and medicinal composition, in accordance with an embodiment of the present invention;

FIG. 10 illustrates a perspective view of an exemplary dual function sterilization seal and label that combines the labeled seal and the second label, in accordance with an embodiment of the present invention;

FIG. 11 illustrates a perspective view of the dual function sterilization seal and label with the labeled seal and the second label detached from the vial, in accordance with an embodiment of the present invention;

FIG. 12 illustrates a side view of a spike having a third label that attaches to the inside of the spike container, in accordance with an embodiment of the present invention;

FIG. 13 illustrates a side view of the spike having the third label on the inside of the spike container and a syringe, in accordance with an embodiment of the present invention;

FIGS. 14A and 14B illustrate side view of labels on a vial, where FIG. 14A shows a sticker label and FIG. 14B shows a string label attached to the neck of the vial, in accordance with an embodiment of the present invention;

FIG. 15 illustrates a side view of a label on an ampule, in accordance with an embodiment of the present invention;

FIG. 16 illustrates a sectioned view of an inner label on a spike cover, in accordance with an embodiment of the present invention;

FIGS. 17A, 17B, and 17C illustrate three different styles of syringes with labels, where FIG. 17A shows a syringe with marks and a clip, FIG. 17B shows a syringe with a sticker label, and FIG. 17C shows a syringe with an elongated neck, in accordance with an embodiment of the present invention; and

FIG. 18 illustrates an alternative embodiment of the system for associating a syringe with a corresponding vial and medicinal composition, showing spike packaging that is substantially the same color as the label, in accordance with an embodiment of the present invention.

Like reference numerals refer to like parts throughout the various views of the drawings.

### DETAILED DESCRIPTION OF THE INVENTION

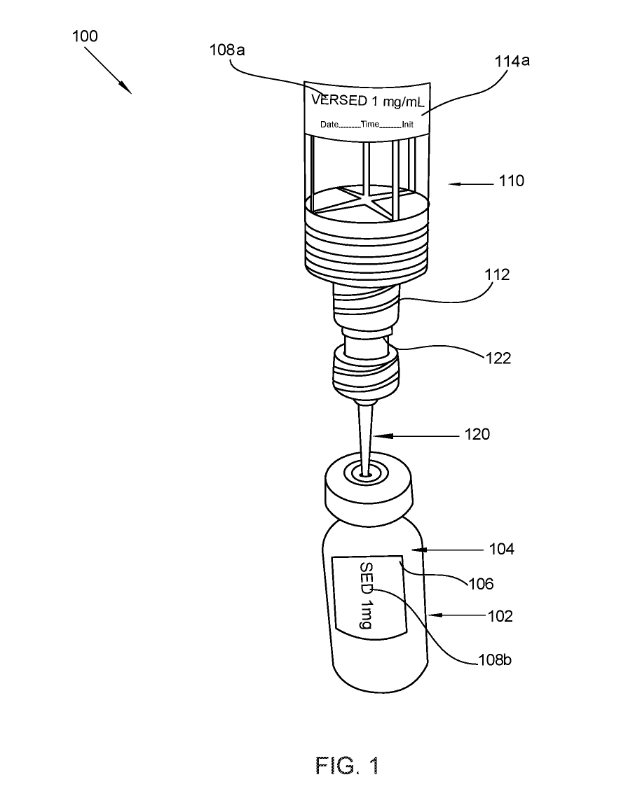

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A system 100 and method 200 for associating a syringe with a corresponding vial and medicinal composition is referenced in FIGS. 1-18. In some embodiments, the system 100 for associating a syringe with a corresponding vial and medicinal composition, hereafter “system 100”, may be configured to associate a syringe 110 and an attached spike 120 with a corresponding vial 102 and the medicinal composition 104 contained in the vial 102.

The system 100 may include a labeled seal 114a, 114b, 114c that substantially encapsulates a spike 120. The labeled seal 114a, 114b, 114c may be detached from the spike 120 for attachment to the syringe 110. The syringe 110 and spike 120 may then be coupled together for administering the medicinal composition 104.

In some embodiments, the labeled seal 114a, 114b, 114c may be defined by at least one identifier 108a, 108b, 108c. The identifier may include, without limitation, a colored panel 126, a tactile panel 128, and a textual indicia panel 130, color, texture, geometric designs, and text. The at least one identifier 108a, 108b, 108c that forms on the labeled seal 114a, 114b, 114c serves to match a similar identifier 108a, 108b, 108c s on a vial label 106 that attaches to a vial 102 that contains the medicinal composition 104. Thus, a matching identifier 108a, 108b, 108c on the syringe 110 and the vial label 106 indicates that the syringe 110 and attached spike 120 are operational with the medicinal composition 104 contained in the vial 102.

This also indicates the type of medicinal composition 104 in the vial 102. The identifier 108a, 108b, 108c on the labeled seal 114a, 114b, 114c is also indicative of the medicinal composition 104 in the vial 102. For example, a color panel that is white may indicate Pitocin. In this manner, the matching identifier 108a, 108b, 108c s on the labeled seal 114a, 114b, 114c and the vial label 106 enable association between the syringe 110 and the vial 102, so as to utilize the appropriate medicinal composition 104 with the syringe 110.

Furthermore, a second label 124 is used to provide additional identification means and ensure that a medical practitioner does not neglect to label the syringe. The second label 124 is configured to secure between the syringe 110 and spike 120. Similar to the labeled seal 114a, 114b, 114c, the second label 124 has at least one identifier 108a, 108b, 108c and serves to match the syringe 110 with the corresponding vial 102 and medicinal composition 104 contained therein.

As referenced in FIG. 1, the system 100 for associating a syringe 110 with a corresponding vial 102 and medicinal composition 104, comprises a vial 102 containing a medicinal composition 104. The vial 102 may include a drug vial or a small glass or plastic vessel or bottle, known in the art for storing medication, such as liquids, powders, and capsules. The vial 102 may contain a medicinal composition 104, including, without limitation, Pitocin, Propofol, Versed, Atropine, and Sublimaze. Those skilled in the art will recognize that each medicinal composition 104 must remain segregated within the vial 102 and used with only a single syringe 110 and spike 120, so as to avoid contamination.

Those skilled in the art will recognize that in many medical procedures, several different medicinal fluids are utilized in connection with the care and treatment of a patient. Some medicinal fluids have similar remedial or chemical properties, whereas others can have quite different remedial effects. In some cases the use or mixing of medicinal fluids have divergent qualities that can be dangerous to the patient.

In one embodiment, the vial 102 is glass and has a generally tubular shape. The vial 102 may also have a bottle-like shape with a neck. However in other embodiments, the vial 102 is a glass vial with a screw cap. The vial 102 may also be a lip vial (closed with a cork or plastic stopper), or a crimp vial (closed with a rubber stopper and a metal cap). Additionally, the vial 102 may be a plastic vial, which can be molded in plastic, can have other closure systems, such as hinge caps which snap shut when pressed.

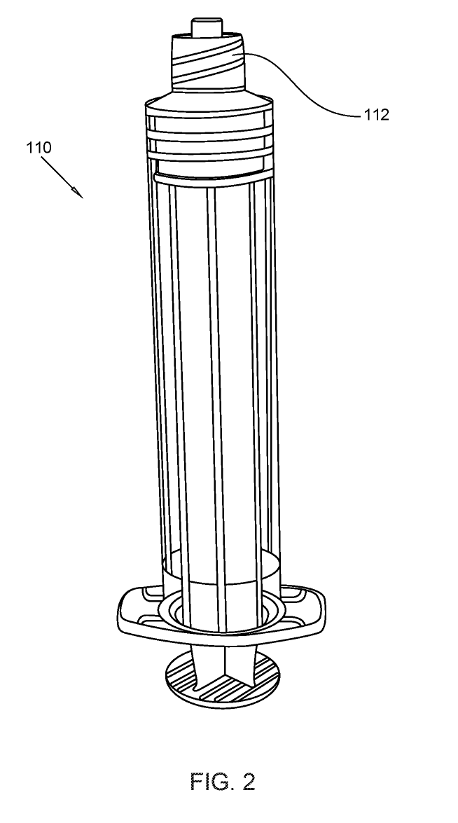

Looking now at FIG. 2, the system 100 includes a syringe 110 having a plunger, a piston, and a male lock fitting 112. The syringe 110 may include a medical style syringe known in the art and having a tubular shape with a nozzle and piston for sucking in and ejecting a liquid form of the medicinal composition 104 in a thin stream. This liquid stream may be used for cleaning wounds or body cavities, or fitted with a spike 120 (hollow needle) for injecting or withdrawing fluids or medicinal composition 104 from the vial 102.

Those skilled in the art will recognize that medical syringes play a vital role in the treatment and care of patients in medical environments. Medical syringes provide a quick, easy and relatively unobtrusive means for administering medicinal fluids to a patient's body during a medical procedure.

As shown in FIG. 3, the system 100 may further include a spike 120 that cooperates with the syringe 110 for administrating the medicinal composition 104. The spike 120 detachably attaches to the syringe 110 for operational use. The spike 120 may include a cooperating female lock fitting 122 adapted to couple to the male lock fitting 112 of the syringe 110. The male lock fitting 112 and the female lock fitting 122 are threaded and have a rotatable relationship. The male lock fitting may be threaded and form a part of a Luer lock that couples to a female lock fitting on a spike 120, so that the syringe 110 detachably couples to the spike 120, as discussed below.

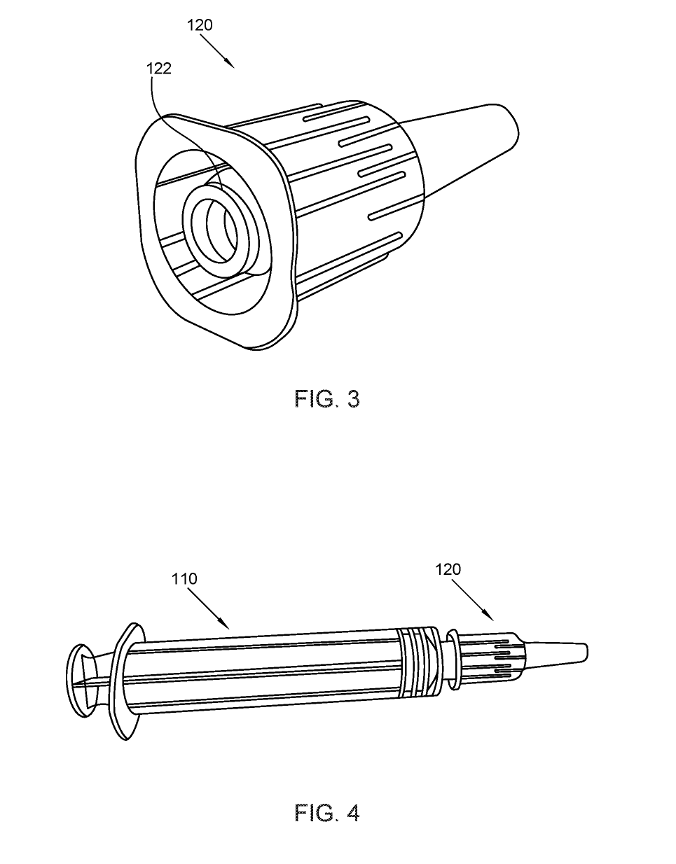

When coupled together, the spike 120 and the syringe 110 are in fluid communication, as shown in FIG. 4. In this manner, the coupled syringe 110 and the spike 120 work to administer the medicinal composition 104. However in other embodiments, other coupling means known in the art may be used to couple the syringe 110 to the spike 120, such as a snap-fit relationship, a screw, or a magnetic connection.

Those skilled in the art will recognize that in order to use the medication, a spike 120, or hollow, tapered needle is affixed to the syringe 110. The plunger of the syringe 110 is withdrawn to a volume that is roughly equivalent to the volume of the liquid medicinal composition 104 that is desired to be transferred to the syringe 110, the needle is used to pierce the cap, or septum of the vial 102 is inverted to place the liquid contents in contact with the spike 120 and the septum. Withdrawal of this volume of liquid medicinal composition 104 equalizes pressure within the vial 102. The spike 120 is then withdrawn from the septum, which re-seals, and the desired subcutaneous injection, or other disposition of the liquid medicinal composition 104 can take place.

In some embodiments, the system 100 comprises multiple spikes having different identifiers and segregated based on the identifier 108a, 108b, 108c in a container. For example, multiple spike 120s are arranged in a box with dividers, separating the spikes by the color of the labeled seal 114a, 114b, 114c that covers each spike. For example, the spikes are segregated so that two hundred spikes have a sealed label with a white colored identifier 108a, 108b, 108c, two hundred spikes have a sealed label with an orange colored identifier 108a, 108b, 108c, two hundred spikes have a sealed label with a yellow colored identifier 108a, 108b, 108c, and two hundred spikes have a sealed label with a blue colored identifier 108a, 108b, 108c.

Looking again at FIG. 1, the system 100 may further include a vial label 106 defined by at least one identifier 108a, 108b, 108c. The vial label 106 is configured to attach, or more specifically, adhere to the vial 102 through an adhesive. The vial label 106 may include a panel having an adhesive that adheres the vial label 106 to an outer sidewall or cap on the vial 102.

In some embodiments, the identifier 108a, 108b, 108c may include, without limitation, a colored panel 126, a tactile panel 128, and a textual indicia panel 130. The identifier 108a, 108b, 108c is configured to provide a visual or tactile means to identify and match the vial 102 and the medicinal composition 104 contained therein, with the appropriate syringe 110 and spike 120. The identifier 108a also indicates the type of medicinal composition 104 in the vial 102, as the medical composition may follow a color-coded scheme.

In one embodiment, the colored panel 126 may include, without limitation, a white color, a yellow color, an orange color, a green color, and a blue color. The color may be associated with a specific medicinal composition 104. For example, white represents Pitocin; yellow represents Propofol; orange represents Versed; green represents Atropine; and blue represents Sublimaze. Thus, the identifier 108a, 108b, 108c in this case serves to not only match the medicinal composition 104 with the appropriate syringe 110, but also to identify the type of medicinal composition 104.

In another embodiment, the tactile panel 128 may include Braille or bumps that indicate the type of medicinal composition 104. For example, a practitioner may feel the bumps from both the labeled seal 114a and the vial label 106 to determine correlation therebetween. The textual indicia panel 130 may simply include large, clear lettering that identifies a specific medicinal composition 104. In any case, the at least one identifier 108a, 108b, 108c matches the medicinal composition 104 with the appropriate syringe 110, and also identifies the type of medicinal composition 104.

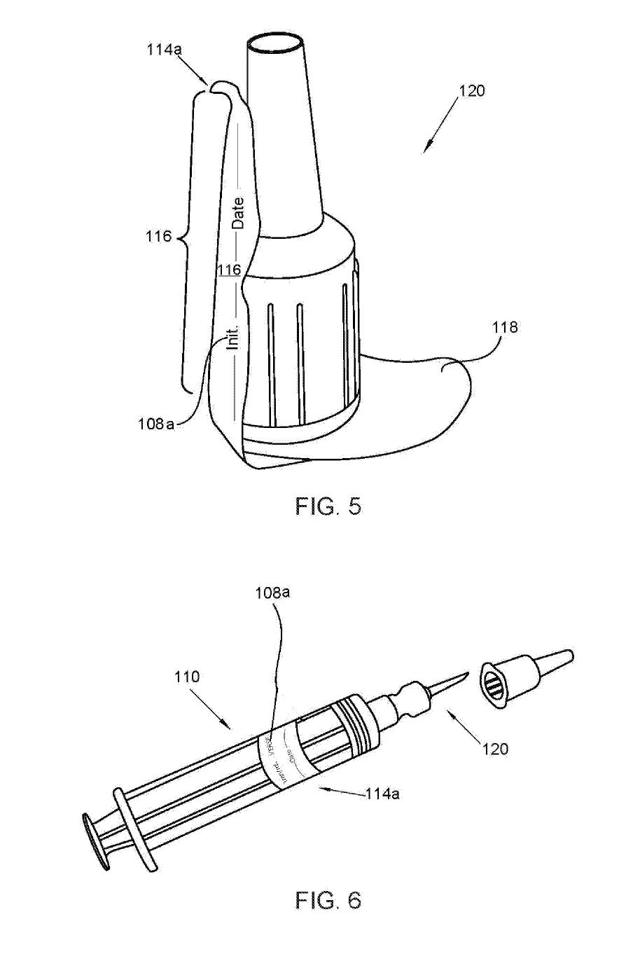

In some embodiments, the system 100 may further include a labeled seal 114a, 114b, 114c that is defined by similar identifiers 108a, 108b, 108c as found in the vial label 106. In this manner, the labeled seal 114a, 114b, 114c associates the syringe 110 to a corresponding vial 102 based on the at least one identifier 108a, 108b, 108c. In one embodiment, the labeled seal 114a, 114b, 114c detachably attaches, or, encapsulates, the spike 120. The labeled seal 114a may be removed from the spike 120, or a rolled configuration, and easily adhered to the syringe 110 for identification purposes. Thus, the labeled seal 114a, 114b, 114c easily detaches from the spike 120 and reattaches to the syringe 110, as shown in FIG. 6. The practitioner does not have to make multiple steps of walking to a dispensary, as the labeling process is performed immediately with the spike 120 and syringe 110.

The labeled seal 114a, 114b, 114c may include a cover 116 that substantially encapsulates the spike 120 (FIG. 5). The labeled seal 114a, in the form of a cover 116 may be sterile and smooth, so as to minimize introduction of bacteria on the surface. In some embodiments, the cover 116 embodiment of the labeled seal 114a may include an adhesive portion 118 that enables attachment to the spike 120, and subsequently, the syringe 110.

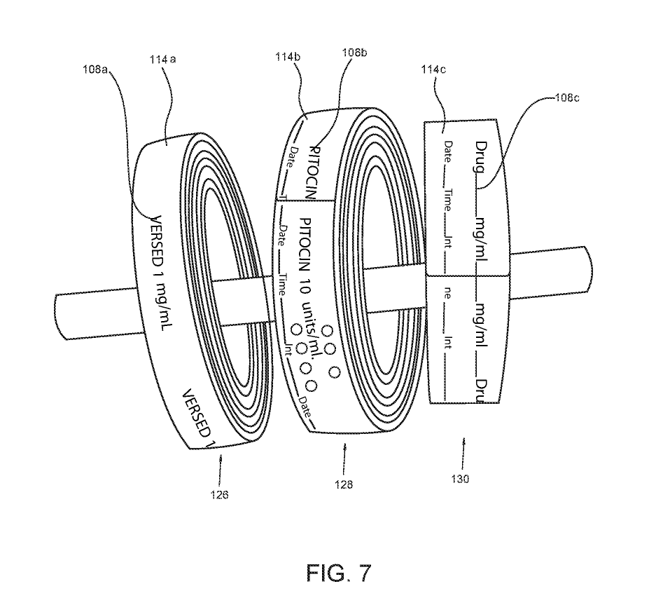

Turning now to FIG. 7, the labeled seal 114a, 114b, 114c may be adapted to form a rolled configuration, and to be perforated into multiple equally-sized segments. In this manner, equal, smooth-edged segments of labeled seal 114a, 114b, 114c may be torn from the roll of labeled seal 114a, 114b, 114c s along a perforated line for attachment to the spike 120 or syringe 110.

Thus, when the medical practitioner needs to couple the spike 120 to the syringe 110, the medical practitioner can rip the labeled seal 114a, 114b, 114c off the roll and stick it directly onto the syringe 110. This is useful in that: it saves the material used to make the labeled seal 114a, i.e., white adhesive cover, and it saves the medical practitioner time, as this eliminates the step where the medical practitioner needs to walk to a dispenser to obtain a labeled seal 114a having the appropriate identifier 108a.

For example, the labeled seal 114a, 114b, 114c covers the spike 120 until operational needs, at which time the labeled seal 114a is removed from the spike 120 and adhered to the syringe 110, as illustrated in FIG. 6. In this manner, the labeled seal 114a, 114b, 114c associates the syringe 110 to a corresponding vial 102 based on the at least one identifier 108a, 108b, 108c. Thus if the identifier 108c is green, the syringe 110 should be used with a vial 102 having a green vial label 106, thereby indicating Atropine.

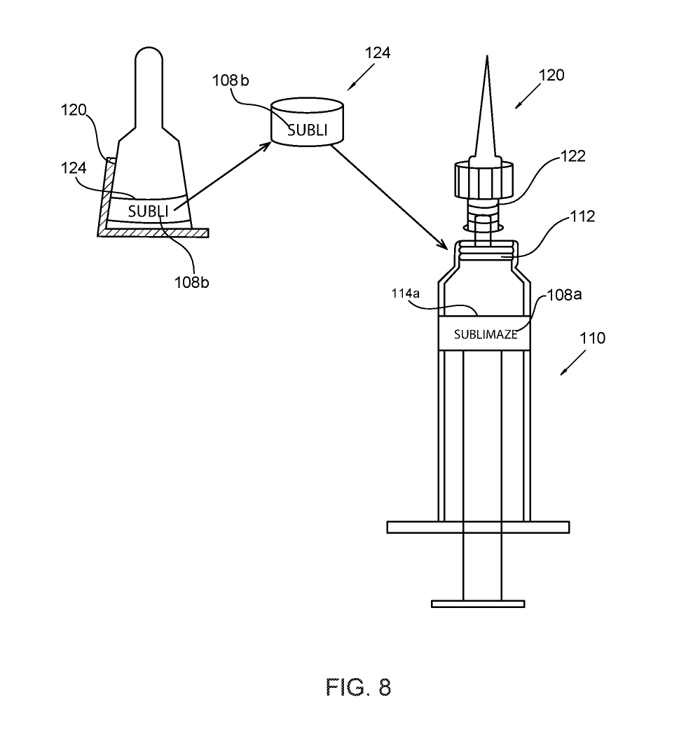

As referenced in FIG. 8, the system 100 may further include a second label 124 defined by the identifier 108b. The second label 124 attaches to the syringe 110, or the spike 120, or both. The second label 124 is effective for associating the syringe 110 with the corresponding vial 102 based on the identifier 108a, 108b.

In this manner, the second label 124 provides an additional layer of labeling security through use of a second label 124 that is secured in a Luer lock between the male lock fitting 112 of the syringe 110, and the female lock fitting 122 of the spike 120. In some embodiments, the second label 124 is a ring that fits around the neck of the male and female lock fittings 112, 122 between the syringe 110 and spike 120. Though in other embodiments, the second label 124 may utilize adhesion means or friction fit means to attach.

FIG. 9 illustrates a flowchart of an exemplary method 200 for associating a syringe with a corresponding vial and medicinal composition, the method comprising an initial Step 202 of providing a vial containing a medicinal composition. The vial 102 may contain a medicinal composition 104, including, without limitation, Pitocin, Propofol, Versed, Atropine, and Sublimaze.

A Step 204 comprises attaching a vial label to the vial. The vial label 106 is defined by at least one identifier. The at least one identifier 108a. 108b, 108c includes at least one of the following: a colored panel, a tactile panel, and a textual indicia panel.

In some embodiments, a Step 206 may include providing a syringe. The syringe 110 may include a medical style syringe known in the art and having a tubular shape with a nozzle and piston for sucking in and ejecting a liquid form of the medicinal composition 104 in a thin stream. This liquid stream may be used for cleaning wounds or body cavities, or fitted with a spike 120 (hollow needle) for injecting or withdrawing fluids or medicinal composition 104 from the vial 102.

A Step 208 includes providing a spike configured to detachably attach to the syringe for administering the medicinal composition. The spike 120 cooperates with the syringe 110 for administrating the medicinal composition 104. The spike 120 detachably attaches to the syringe 110 for operational use. The spike 120 may include a cooperating female lock fitting 122 adapted to couple to the male lock fitting 112 of the syringe 110. The male lock fitting 112 and the female lock fitting 122 are threaded and have a rotatable relationship.

A Step 210 comprises providing a labeled seal, the labeled seal defined by the at least one identifier, the labeled seal configured to detachably attach to the spike or the syringe. The labeled seal 114a, 114b, 114c may be a cover 116 that substantially encapsulates the spike 120 (FIG. 5). The cover 116 may be sterile and smooth, so as to minimize introduction of bacteria on the surface. The cover 116 may include an adhesive portion 118 that enables attachment to the spike 120, and subsequently, the syringe 110.

A Step 212 may include detaching the labeled seal from the spike. In one embodiment, the labeled seal 114a, 114b, 114c detachably attaches, i.e., encapsulates, the spike 120 and adheres to the syringe 110. In this manner, the labeled seal 114a, 114b, 114c easily detaches from the spike 120 and reattaches to the syringe 110, as shown in FIG. 6. In some embodiments, the method 200 may include a Step 214 of attaching the labeled seal to the syringe, whereby the labeled seal associates the syringe to a corresponding vial based on the at least one identifier.

A Step 216 includes providing a second label, the second label defined by the at least one identifier, the second label configured to detachably attach to the spike, or the syringe, or both, whereby the second label associates the syringe to the corresponding vial based on the at least one identifier. The second label 124 is effective for associating the syringe 110 with the corresponding vial 102 based on the at least one identifier 108a, 108b.

In this manner, the second label 124 provides an additional layer of labeling security through use of a second label 124 that is secured in a Luer lock between the male lock fitting 112 of the syringe 110, and the female lock fitting 122 of the spike 120. A Step 218 comprises administering the medicinal composition from the vial having a matching identifier with the syringe.

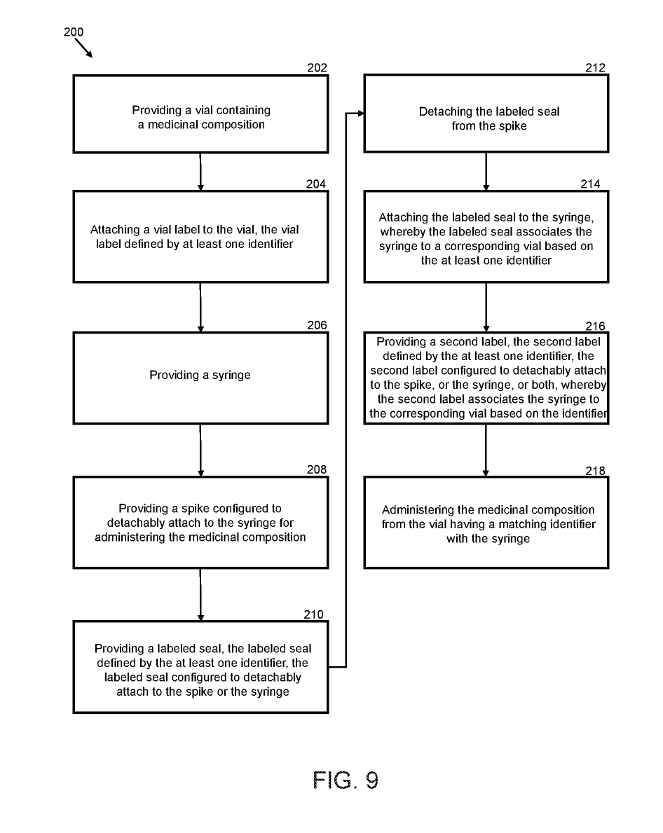

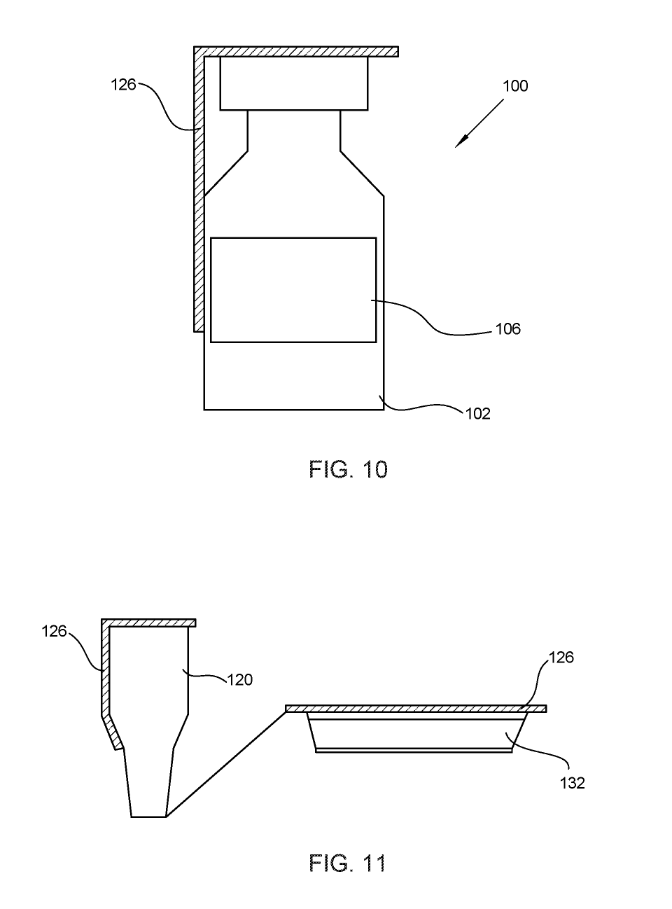

FIG. 10 illustrates an alternative embodiment of the system 100, showing a perspective view of an exemplary dual function sterilization seal and label 126 on the vial 102. The dual function sterilization seal and label combines the labeled seal 114a that detachably attaches to the spike 120, with the second label 124 and attaches to the syringe 110 and the spike 120. Thus, the dual function sterilization seal and label 126 attaches to both the vial 102 and the syringe 110. The use of a dual function labeling component reduces the amount of material needed to label the syringe 110. Also, the user can choose which labels, between the labeled seal 114a-c, the second label 124, and the dual function sterilization seal and label to use.

As shown in FIG. 11, the dual function sterilization seal and label 126 may utilize an adhesive on an underside for attachment to a vial cap 132 that fits over an opening in the vial 102. The adhesive also maintains attachment with the spike 120. Thus, the dual function sterilization seal and label 126 is operable with the vial 102, the vial cap 132, and the spike 120. In one embodiment, the dual function sterilization seal and label 126 may be operable on a spike that is flat and rectangular, on which the dual function sterilization seal and label 126 lies coplanar. Similar to the labeled seal 114a-c and the second label 124, the dual function sterilization seal and label 126 may be defined by the at least one identifier 108a-c for identifying means.

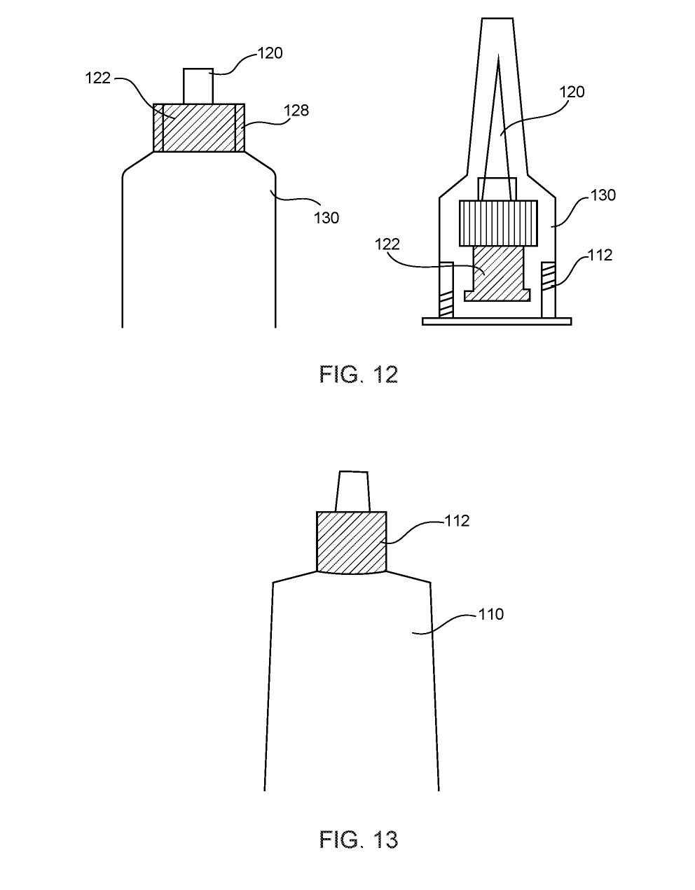

Looking now at FIG. 12, the system 100 also provides an alternative third label 128 option that provides the user with more labeling possibilities in addition to the labeled seal 124, the second label 124, and the dual function sterilization seal and label 126 discussed above. The third label 128 attaches to the inside of a spike container 130, or the housing in which the spike is retained until use. The third label 128 attaches to the inside of the spike container 130, engaging the Luer lock of the syringe 110 when an operator pushes in and rotates the male lock fitting 112 of the syringe 110 with the female lock fitting 122 of the spike 120. FIG. 13 illustrates the syringe 110 attached with the spike 120 in this manner, and the third label 128 visible from the junction therebetween. Similar to the labeled seal 114a-c and the second label 124, the third label 128 may be defined by the at least one identifier 108a-c for identifying means.

It is significant to note that the medical industry utilizes myriad styles and sizes of syringes and spikes. The present invention attempts to associate a syringe or other medical instrument with a corresponding vial and the medicinal composition contained in the vial. This can be used with the various styles and sizes of syringes, ampules, vials, and containers. The labeling for each type of medical instrument must be easily viewed, and as described above, easy to remove from the spike and attached to the syringe.

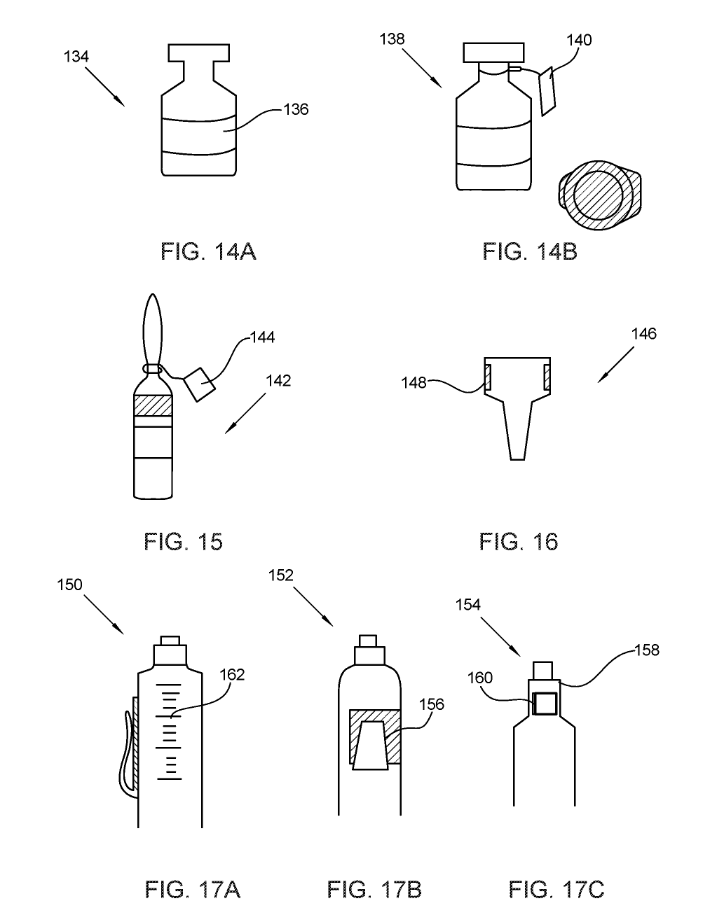

For example, FIGS. 14A and 14B illustrate side views of labels on a first vial 134 and a second vial 138. The first vial 134 utilizes a sticker label 136, while the second vial 138 utilizes a string label 140 attached to the neck of the vial 138. Looking now at an ampule 142, shown in FIG. 15, an inner label 144 is affixed to an inner surface of the ampule 142. The inner label 144 may be viewed through a semi-transparent material that the ampule 142 is fabricated from. The inner label 144 here is colored and labeled, as described above. Turning now to FIG. 16, a spike cover 146 also utilizes an inner label 148 through a semi-transparent surface material.

FIGS. 17A, 17B, and 17C illustrate three different styles of syringes 150, 152, 154 with an outer label 156 and a neck label 158 for an elongated neck 160. In this example, as in above, the label on the seal associates the syringe 150, 152, 154 to a corresponding vial. FIG. 17A shows a syringe 150 with medicine volume marking 162 and a clip. FIG. 17B shows a syringe 152 with an outer sticker label 156 that is colored and has indicia indicating the type of medicinal composition. FIG. 17C shows a syringe 154 with an elongated neck 158. Here, a neck label 160 is affixed to the elongated neck 158. However in other embodiment, other types of syringes.

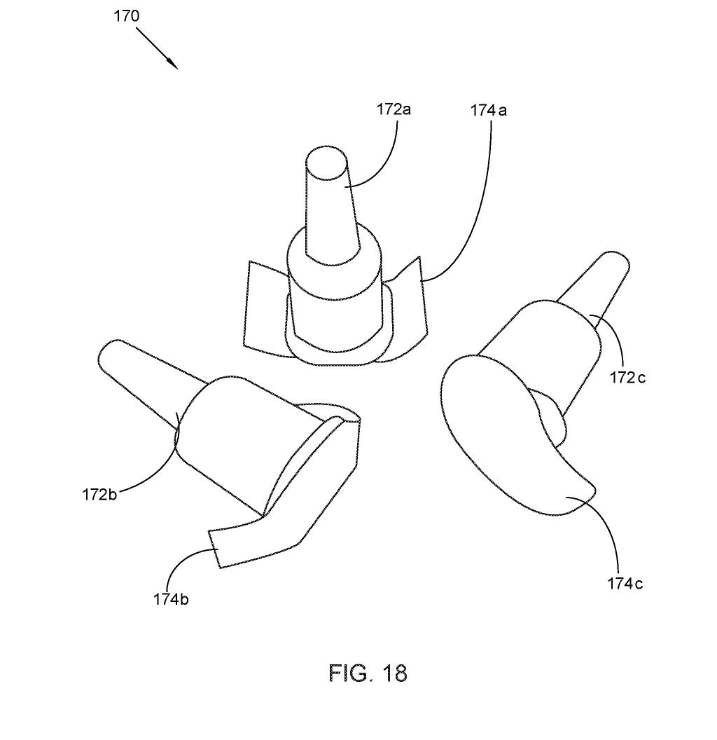

FIG. 18 illustrates an alternative embodiment of a system 170 for associating a syringe with a corresponding vial and medicinal composition, showing spike packaging 172a, 172b, 172c that is substantially the same color as a label 174a, 174b, 174c. The use of the same color for the spike 172a-c and the label 174a-c can be advantageous for quicker identification of the medicinal composition. For example, for the spike 172a to be used with the medicinal composition, Sublimaze, the spike 172a and the label 174a are both defined by a blue color. Though any combination of coloring with the spike 172a-c, the label 174a-c, and the medicinal composition is possible in this alternative embodiment of the system 170.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A system for associating a syringe with a corresponding vial and medicinal composition, the system comprising:
a vial containing a medicinal composition;
a syringe;
a spike adapted to couple to the syringe,
whereby the coupled syringe and the spike work to administer the medicinal composition;
a vial label defined by at least one identifier, the vial label being attached to the vial; and
a labeled seal defined by the at least one identifier, the labeled seal being detachably attached to the spike or the syringe,
whereby the labeled seal associates the syringe to a corresponding vial based on matching of the at least one identifier.

2. The system of claim 1, further comprising a second label defined by the at least one identifier, the second label being attached to the syringe, or the spike, or both.

3. The system of claim 2, wherein the second label associates the syringe to the corresponding vial based on matching of the at least one identifier.

4. The system of claim 1, wherein the at least one identifier includes at least one of the following: a colored panel, a tactile panel, and a textual indicia panel.

5. The system of claim 4, wherein the colored panel includes at least one of the following: a white color, a yellow color, an orange color, a green color, and a blue color.

6. The system of claim 5, wherein the medicinal composition includes at least one of the following: Pitocin, Propofol, Versed, Atropine, and Sublimaze.

7. The system of claim 6, wherein the tactile panel includes Braille.

8. The system of claim 1, wherein the syringe comprises a male lock fitting.

9. The system of claim 8, wherein the spike comprises a cooperating female lock fitting adapted to couple to the male lock fitting of the syringe.

10. The system of claim 9, wherein the male lock fitting and the female lock fitting are threaded and have a rotatable relationship.

11. The system of claim 10, wherein the male lock fitting and the female lock fitting comprise a Luer lock.

12. The system of claim 1, wherein the labeled seal is configured to be perforated.

13. The system of claim 1, wherein the labeled seal is adapted to form a rolled configuration.

14. The system of claim 1, wherein the labeled seal comprises a cover that is sized and dimensioned to substantially encapsulate the spike.

15. The system of claim 14, wherein the cover comprises an adhesive portion.

16. A system for associating a syringe with a corresponding vial and medicinal composition, the system consisting of:
a vial containing a medicinal composition;
a syringe comprising a threaded male lock fitting;
a spike comprising a cooperating threaded female lock fitting adapted to couple to the threaded male lock fitting of the syringe,
whereby the coupled syringe and the spike work to administer the medicinal composition;
a vial label defined by at least one identifier, the at least one identifier including at least one of the following: a colored panel, a tactile panel, and a textual indicia panel, the vial label being attached to the vial;
a labeled seal defined by the at least one identifier, the labeled seal being detachably attached to the spike or the syringe, the labeled seal being adapted to form a rolled configuration, the labeled seal being perforated,
whereby the labeled seal associates the syringe to a corresponding vial based on matching of the at least one identifier; and
a second label defined by the at least one identifier, the second label being attached to the syringe, or the spike, or both,
whereby the second label associates the syringe to the corresponding vial based on matching of the at least one identifier.

17. The system of claim 16, wherein the colored panel includes at least one of the following: a white color, a yellow color, an orange color, a green color, and a blue color.

18. The system of claim 16, further comprising a dual function sterilization seal and label.

19. The system of claim 16, further comprising a third label detachably attached to an inside of a spike container.

20. A method for associating a syringe with a corresponding vial and medicinal composition, the method comprising:
providing a vial containing a medicinal composition;
attaching a vial label to the vial, the vial label defined by at least one identifier;
providing a syringe;
providing a spike configured to detachably attach to the syringe for administering the medicinal composition;
providing a labeled seal, the labeled seal defined by the at least one identifier, the labeled seal configured to detachably attach to the spike or the syringe;
detaching the labeled seal from the spike;
attaching the labeled seal to the syringe, whereby the labeled seal associates the syringe to a corresponding vial based on the at least one identifier;
providing a second label, the second label defined by the at least one identifier, the second label configured to detachably attach to the spike, or the syringe, or both, whereby the second label associates the syringe to the corresponding vial based on the at least one identifier; and
administering the medicinal composition from the vial having a matching identifier with the syringe.
